(19) United States
(12) Patent Application Publication
(10) Pub. No.: US 2026/0168516 A1
(43) Pub. Date: Jun. 18, 2026

(54) POWERED AIR PURIFYING FACESHIELD

(71) Applicant: Board of Regents of the University of Nebraska, Lincoln, NE (US)

(72) Inventors: Elizabeth Beam, Omaha, NE (US); Evan Marsh, Lincoln, NE (US)

(21) Appl. No.: 19/117,546

(22) PCT Filed: Oct. 13, 2023
(86) PCT No.: PCT/US2023/076783

§ 371 Date: Apr. 1, 2025

Related U.S. Application Data
(60) Provisional application No. 63/416,060, filed on Oct. 14, 2022.

(51) Int. Cl. F04D 29/52; A61M 16/00; F04D 25/08; F04D 25/16; F04D 29/44

(52) U.S. Cl. CPC F04D 29/522; A61M 16/0066; F04D 25/08; F04D 25/166; F04D 29/444

(57) ABSTRACT

A powered air purifying faceshield (PAPF) includes a headgear with a face shield frame pivotally coupled to the headgear. The face shield frame is configured to support a face shield that is mounted to the face shield frame. The PAPF further includes a battery powered fan assembly mounted above the face shield frame. The battery powered fan assembly is configured to direct ambient air through an air filter before the air is blown into a region behind the face shield. The PAPF further includes a neck shroud that is coupled to the face shield frame. The neck shroud may extend from a lower edge of the face shield frame. The neck shroud includes one or more filtered exhaust ports for releasing air from the region behind the face shield.

### CROSS-REFERENCE TO RELATED APPLICATIONS

The present application claims priority under 35 U.S.C. § 119(e) to U.S. Provisional Application Ser. No. 63/416,060, filed Oct. 14, 2022, and titled “Healthcare Powered Air Purifying Respirator,” which is incorporated herein by reference in its entirety.

### STATEMENT OF GOVERNMENT SUPPORT

This invention was made with U.S. government support under grant number ED19HDQ0200064 awarded by the Economic Development Administration. The U.S. government has certain rights in the invention.

### TECHNICAL FIELD

The present disclosure generally relates to respiratory protective equipment (RPE), more specifically to powered air purifying respirators (PAPRs).

### BACKGROUND

For healthcare workers, the head is the most vulnerable part of the body. Respiratory protection is used to protect mucous membranes (eyes, nose, mouth) from exposure during aerosol generating procedures as well as those working with patients suffering from COVID-19 and other airborne transmission pathogens. The COVID-19 pandemic also increased awareness of the need for source control using RPE (protecting the patients from healthcare workers).

Healthcare workers typically cover their heads with face shields and filtering facepiece respirators (FFR), such as N95 respirators. However, extended use of FFRs can be very hard on facial skin, and fogging can be a real challenge with face shields. Healthcare workers may instead use powered air purifying respirators (PAPRs) that require both power and filtration in some way. PAPRs offer greater protection than FFRs and do not have to be worn tightly over the mouth and nose region of the face, but they tend to be noisy, heavy, cumbersome, and much more expensive. Most PAPRs also require extensive training and education to use correctly. PAPRs may also have very limited portability as most systems are bulky and must be plugged into a wall outlet for recharging of the motor or batteries. Current PAPR systems can be difficult to don and doff. They create a lot of waste with disposable hoods and tubing when used for isolation. Cleaning flimsy material can also be difficult for extended use in pandemic care.

### SUMMARY

A portable, lightweight, loose-fitting powered air purifying faceshield (PAPF) is disclosed herein. The PAPF solves many of the problems associated with existing systems because of its robust, easy to wear, and easy to use design. The PAPF provides respiratory protection for healthcare workers while also preventing the spread of infection from asymptomatic workers with source control and facilitating improved healthcare provider-patient interactions with full face visibility (able to read lips, expressions). The PAPF may also be utilized in a variety of clinical and ambulatory care settings by healthcare workers to provide respiratory protection and prevent the spread of pathogens and other hazardous materials.

In embodiments, the PAPF includes a headgear with a face shield frame pivotally coupled to the headgear. The face shield frame is configured to support a face shield that is mounted to the face shield frame. The PAPF further includes a battery powered fan assembly mounted above the face shield frame. The battery powered fan assembly is configured to direct ambient air through an air filter before the air is blown into a region behind the face shield. The PAPF further includes a neck shroud that is coupled to the face shield frame and snug to the user's chin/face. The neck shroud may extend from a lower edge of the face shield frame. The neck shroud includes one or more filtered exhaust ports for releasing filtered air from the region behind the face shield.

This Summary is provided solely as an introduction to subject matter that is fully described in the Detailed Description and Drawings. The Summary should not be considered to describe essential features nor be used to determine the scope of the Claims. Moreover, it is to be understood that both the foregoing Summary and the following Detailed Description are example and explanatory only and are not necessarily restrictive of the subject matter claimed.

### BRIEF DESCRIPTION OF THE DRAWINGS

The Detailed Description is provided with reference to the accompanying Drawings. The use of the same reference numbers in different instances in the Detailed Description and the Drawings may indicate similar or identical items. The Drawings are not necessarily to scale, and any disclosed processes may be performed in an arbitrary order, unless a certain order of steps/operations is inherent or specified in the Detailed Description or in the Claims.

FIG. 1 is a right side perspective view of a powered air purifying faceshield (PAPF), in accordance with one or more embodiments of this disclosure.

FIG. 2 is a left side perspective view of the PAPF of FIG. 1, in accordance with one or more embodiments of this disclosure.

FIG. 3 is a left side perspective view of the PAPF of FIG. 1, in accordance with one or more embodiments of this disclosure.

FIG. 4 is a right side perspective view of the PAPF of FIG. 1, in accordance with one or more embodiments of this disclosure.

FIG. 5 is a frontal perspective view of the PAPF of FIG. 1, in accordance with one or more embodiments of this disclosure.

FIG. 6 is a left side perspective view of the PAPF of FIG. 1, wherein the PAPF is placed into a raised (resting/inactive) configuration by rotating a face shield frame of the PAPF about a set of pivots that couple the face shield frame to a headgear worn by a user, in accordance with one or more embodiments of this disclosure.

FIG. 7 is a left side perspective view of the PAPF of FIG. 1, wherein the PAPF is placed into the raised configuration in the same manner as in FIG. 6, in accordance with one or more embodiments of this disclosure.

FIG. 8 is a partially exploded right side perspective view of a PAPF, in accordance with one or more embodiments of this disclosure.

FIG. 9 is a left side perspective view of the PAPF of FIG. 8, in accordance with one or more embodiments of this disclosure.

FIG. 10 is a left side perspective view of the PAPF of FIG. 8, wherein the PAPF is placed into a raised (resting/inactive) configuration by actuating a face shield frame of the PAPF along a set of curved rails that couple the face shield frame to a headgear worn by a user, in accordance with one or more embodiments of this disclosure.

### DETAILED DESCRIPTION

A portable and lightweight powered air purifying faceshield (PAPF) is disclosed herein. The PAPF solves many of the problems associated with existing systems because of its robust, easy to wear, and easy to use design. The PAPF provides respiratory protection for healthcare workers while also preventing the spread of infection from asymptomatic workers with source control and facilitating improved healthcare provider-patient interactions (full face visibility for lip reading and expressions). The PAPF may also be utilized in a variety of settings by other types of users to provide respiratory protection and prevent the spread of pathogens and other hazardous particles. Examples include, but are not limited to, material manufacturing, production, development, and refining. In addition, certain embodiments of the PAPF may be designed and used for research laboratories handling hazardous pathogens and/or any healthcare environment affected by poor or contaminated air quality.

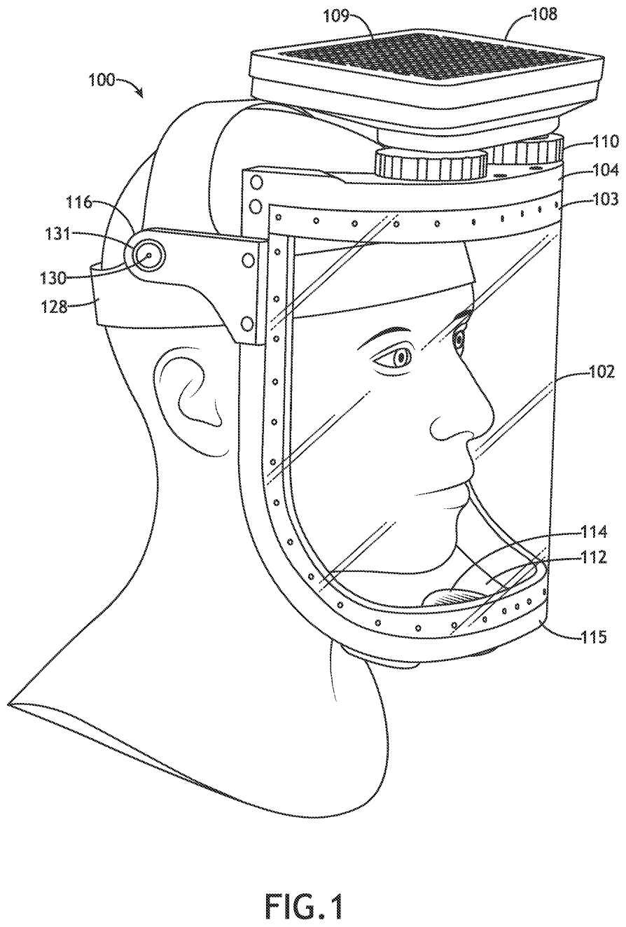

FIGS. 1 through 10 illustrate embodiments of the PAPF, which includes a number of improvements over traditional PAPR designs. Compared with traditional PAPRs, the presently disclosed PAPF may be much easier to use and clean when needed rapidly in clinical care. In certain embodiments, the weight of the PAPF is also much lighter than existing PAPRs, resulting in improved ergonomics. All electronics may be integrated into the PAPF frame to lower decibel production (i.e., reduce noise) and to facilitate improved cleaning and sanitization. The PAPF also includes a headgear with improved ergonomics that attaches the PAPF more securely to a user's head. The PAPF may include a large face shield window to improve the user's vision and allow for improved patient-provider interactions as the patient can more easily see the user's (e.g., healthcare worker's) expressions and even read their lips if necessary.

Example embodiments of the PAPF are discussed below with reference to FIGS. 1 through 10. Those skilled in the art will appreciate that the embodiments illustrated in FIGS. 1 through 10 are specific examples that can be modified by changing dimensions or adding/removing non-essential components or fillers. Furthermore, two or more embodiments (or portions thereof) can be combined to achieve an additional embodiment that is not necessarily shown or described with reference to one drawing. As such, the drawings should not be construed as restrictive of any particular embodiment and are intended instead as visual aids to help describe configurations of certain components that may apply to multiple embodiments of the PAPF.

FIGS. 1 through 7 illustrate a PAPF 100 in accordance with an embodiment of this disclosure. As shown in FIG. 1, the PAPF 100 includes a face shield 102 that is mounted to a face shield frame 103. For example, the face shield 102 may be held between inner and outer frame members of the face shield frame 103, or the face shield 102 may be secured to an inner or outer surface of the face shield frame 103 by a plurality of fasteners, adhesive (e.g., glue), or any other type of fastening mechanism.

In embodiments, the face shield 102 is designed to be larger than a typical user's face profile. For example, the face shield 102 may have a width greater than the user's ear-to-ear distance and a length greater than the user's forehead-to-chin distance. To achieve proper dimensions the face shield can be designed using (National Institute for Occupational Safety and Health) NIOSH Anthropometric Data and ISO Digital Headforms, published by The National Personal Protective Technology Laboratory (NPPTL), updated Apr. 24, 2020.

The PAPF 100 further includes a neck shroud 112 that is coupled to the face shield frame 103. The neck shroud 112 may be made of fluid resistant fabric, scuba rubber, biological suit material, silicone, TPU, or similar flexible material. In embodiments, the neck shroud 112 extends from a lower edge of the face shield frame 103 to seal gaps between a user's head and the face shield frame 103. The neck shroud 112 may include one or more filtered exhaust ports 114 that are configured to expel air from a region behind the face shield 102. In some embodiments, each filtered exhaust port 114 comprises an opening (or transmissive fabric) that includes or is overlaid by a filter for source control. The exhaust port filter material may include, but is not limited to, surgical mask material or any other American Society for Testing Materials (ASTM) level 3 filter material.

The neck shroud 112 may be removably coupled to the face shield frame 103 so that the neck shroud 112 and face shield frame 103 can be disassembled for sanitization and reassembled thereafter. In some embodiments, the neck shroud 112 is removably coupled to the face shield frame 103 by an edge guard 115 that extends along the lower edge of the face shield frame 103. The edge guard 115 may be configured to grasp an upper edge of the neck shroud 112 so that it is held tightly between the edge guard 115 and the lower edge of the face shield frame 103.

In some embodiments, the neck shroud 112 is configured to be coupled to a full body personal protective equipment (PPE) suit (interface of one PPE element with another). For example, the neck shroud 112 may include one or more fasteners (e.g., buttons, buttonholes, snap fasteners, clasps, zipper elements, VELCRO elements, tabs, etc.) that are configured to secure a lower edge of the neck shroud 112 to a collar of a full body PPE suit or simply support safe doffing. Alternatively, the neck shroud 112 may be coupled to the full body PPE suit by simply nesting the lower edge of the neck shroud within the collar and hood of the full body PPE suit.

The PAPF 100 further includes a battery powered fan assembly 104 mounted above the face shield frame 103. The battery powered fan assembly 104 may include any type of electric fan or blower. For example, the battery powered fan assembly 104 may include at least one electric motor that drives a respective fan propeller. In some embodiments, the battery powered fan assembly 104 includes a VR headset fan or similar.

The battery powered fan assembly 104 may be mounted within a compartment that is defined by or coupled to an upper edge of the face shield frame 103. In some embodiments, the battery powered fan assembly 104 is in its own compartment that is removably coupled to the upper edge of the face shield frame 103 by one or more fasteners (e.g., screws, bolts, latches, friction-fit couplings, etc.) so that the components can be disassembled for sanitization and then reassembled thereafter. Any technique known in the art may be used to sanitize PAPF components. For example, components may be washed thoroughly and/or subjected to heat, chemical agents, and/or radiation (e.g., UV light) to sanitize or decontaminate the components.

The battery powered fan assembly 104 is configured to direct ambient air through an air filter 109 before the air is blown into the region behind the face shield 102. In this manner, filtered air is constantly provided to the user. The air flows through the region behind the face shield 102 is eventually released through the one or more exhaust ports 106. The air filter 109 may be located at or adjacent to an air intake vent at the top of the PAPF 100. The battery powered fan assembly 104 may be located between the air filter 109 and the region behind the face shield 102 so that only filtered air enters the battery powered fan assembly 104. This configuration prevents the battery powered fan assembly 104 from being contaminated during use.

In embodiments, the battery powered fan assembly 104 is configured to suction air through the air filter 109 and then blow the air into the region behind the face shield 102 through one or more vents 106. In some embodiments, the vents 106 have a protective covering with small enough openings to prevent the user's hair from being caught in the battery powered fan assembly 104.

The air filter 109 is preferably an N95 level or other high efficiency particulate air (HEPA) filter with splash protection. In some embodiments, the air filter 109 is pleated to improve airflow through the system. The battery powered fan assembly 104 and the air filter 109 may be configured to provide airflow of at least 6 cubic feet per minute (cfm); although less airflow may be appropriate in certain embodiments.

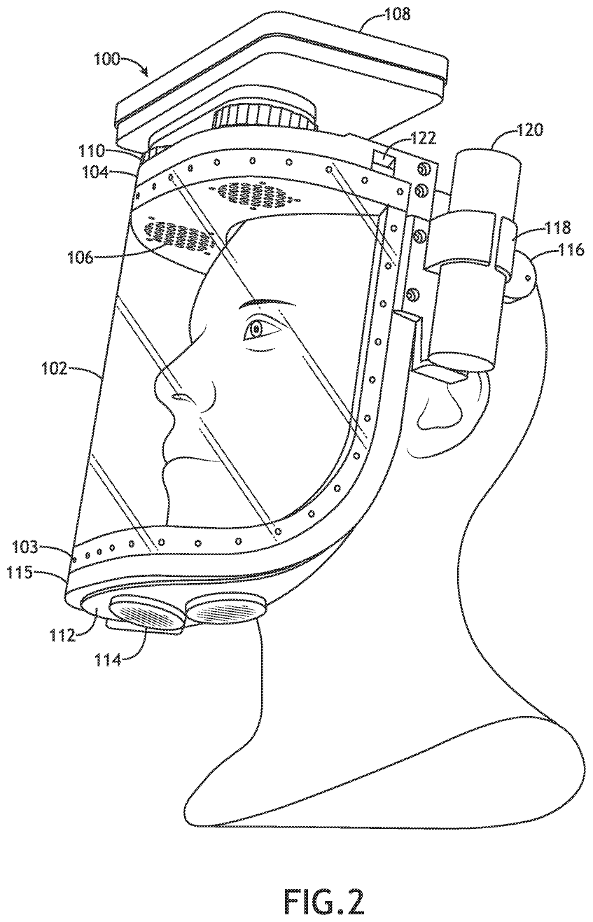

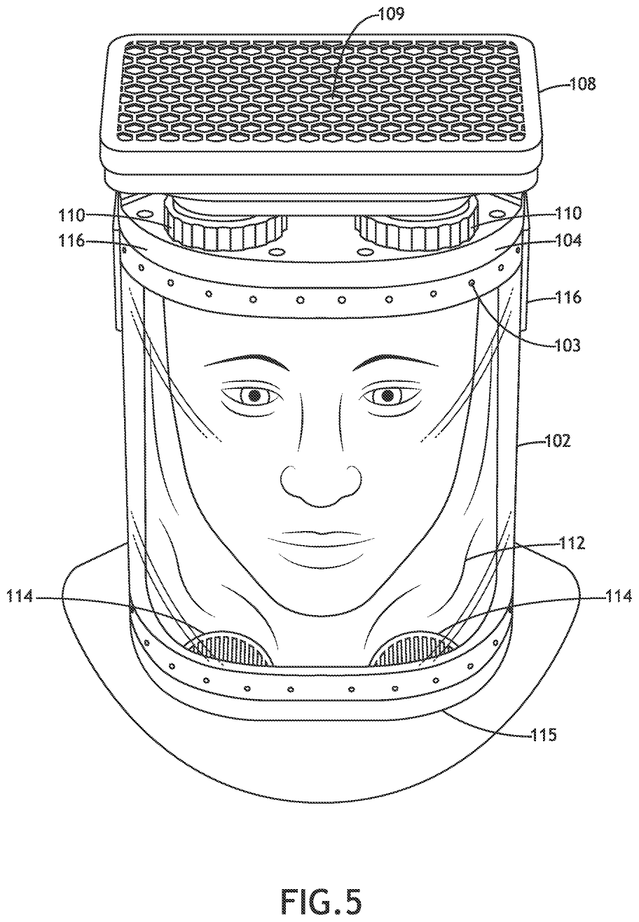

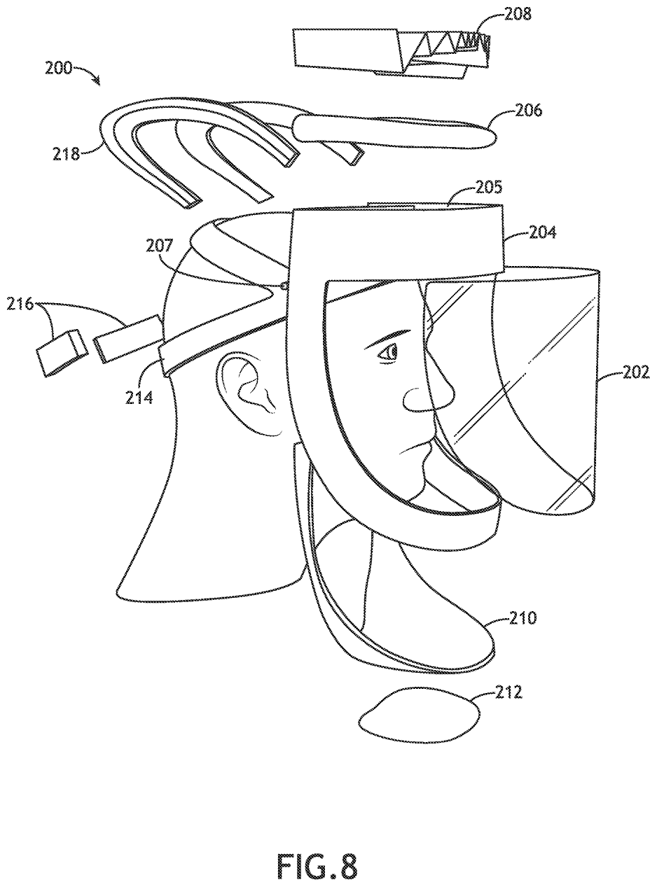

The air filter 109 may be located directly above the battery powered fan assembly 104. For example, the air filter 109 may be mounted within the same compartment as the battery powered fan assembly 104, much like the system illustrated in FIGS. 8 through 10. Although, preferably, as shown in FIG. 2, the air filter 109 is mounted within a separate compartment 108 that is coupled to the compartment of the battery powered fan assembly 104 by one or more air conduits 110. For example, in the embodiment illustrated in FIG. 5, the PAPF 100 includes two air conduits 110 that couple the compartment of the battery powered fan assembly 104 to the separate compartment 108 of the air filter 108. However, any number of air conduits 110 may be appropriate.

The use of air conduits 110 to connect separate compartments of the air filter 109 and the battery powered fan assembly 104 may provide certain advantages for air flow and may reduce noise within the system because of less vibrations as a result of the spacing between the air filter 109 and the battery powered fan assembly 104. The battery powered fan assembly 104 may be configured to generate an operational sound output below 50 decibels (dB); although higher noise output may be appropriate in certain embodiments. The spacing between the air filter 109 and the battery powered fan assembly 104 can also help prevent the battery powered fan assembly 104 from overheating, and the use of separate compartments may help prevent against contamination of the battery powered fan assembly 104.

In some embodiments, the one or more air conduits 110 comprise threaded couplings (e.g., cooperatively threaded tubes and rings) for securing the separate compartment 108 of the air filter 109 to the compartment of the battery powered fan assembly 104. This further allows for components to be disassembled so that they can be sanitized and then reassembled thereafter. It also allows for maintenance and options for repair to sustain a long shelf-life for the device.

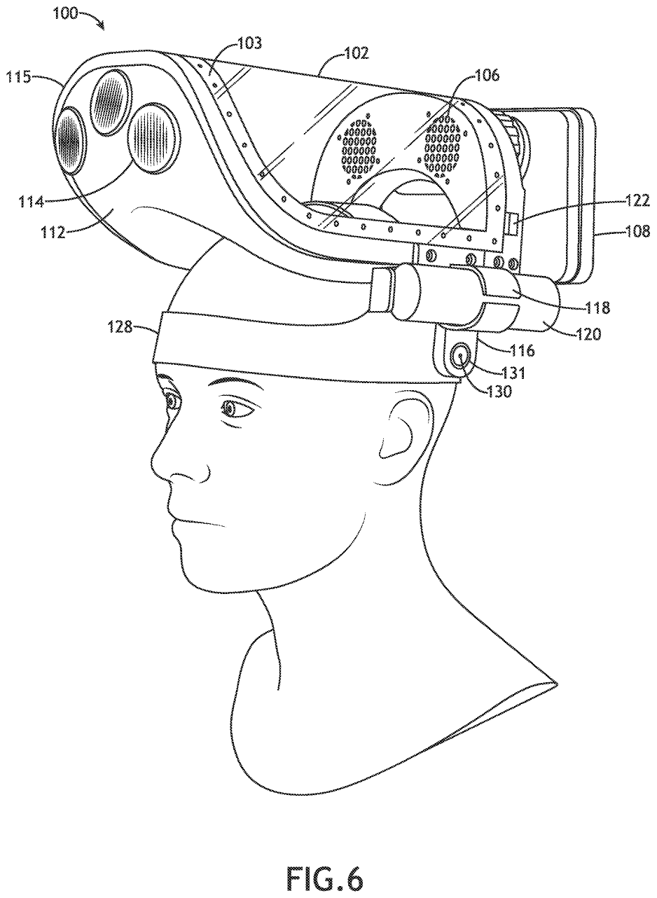

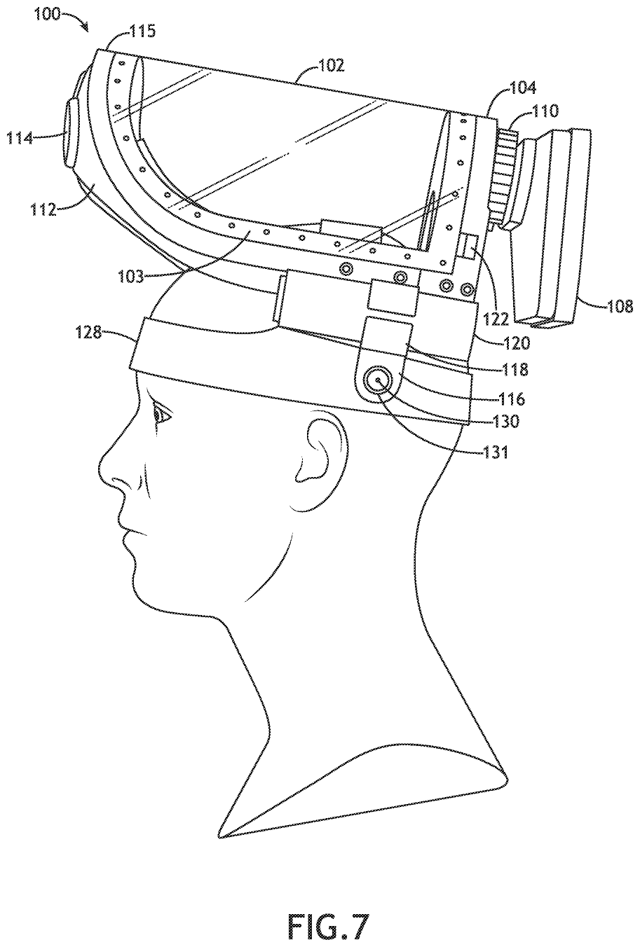

Referring again to FIG. 1, the PAPF 100 includes a headgear 128 configured to support the PAPF components on the user's head. In embodiments, the headgear 128 includes a head strap configured to wrap around the back of the user's head. The headgear 128 preferably wraps entirely around the user's head so that it can hold the face shield frame 103 in a lowered configuration for active use (e.g., as shown in FIG. 1) or in a raised configuration when the PAPF 100 is inactive (e.g., as shown in FIGS. 6 and 7). The headgear 128 may further include an additional strap configured to extend across a top of the user's head for added support. The user can easily don the PAPF 100 by securing the headgear 128 to the user's head. The headgear 128 may include elastic and/or adjustable straps (e.g., adjustable belts) to accommodate different head sizes.

The PAPF 100 is also made from lightweight materials to make it easy to wear for extended periods of time. In preferred embodiments, the PAPF 100 weighs less than 1.5 pounds; although heavier weight materials, hence a heavier PAPF 100, may be appropriate for certain embodiments (e.g., for use in very harsh environments).

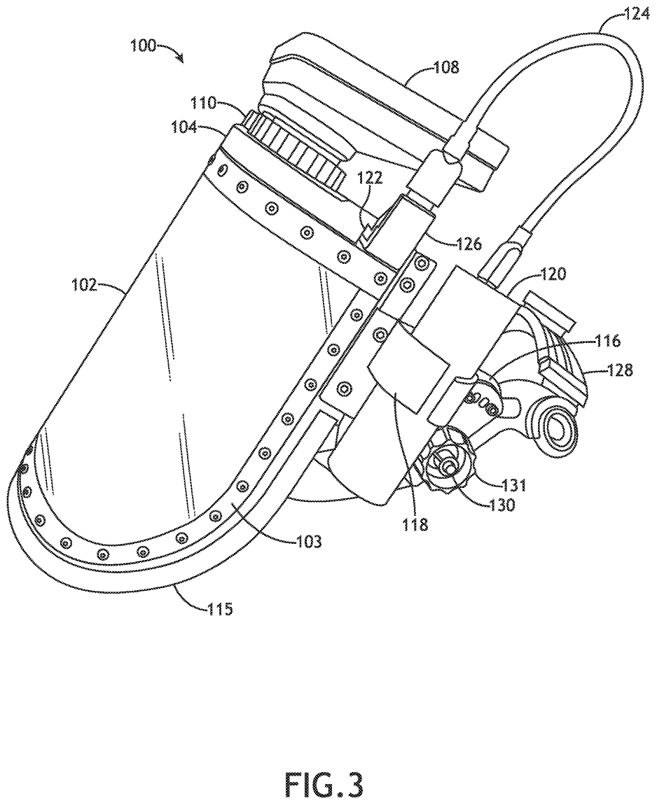

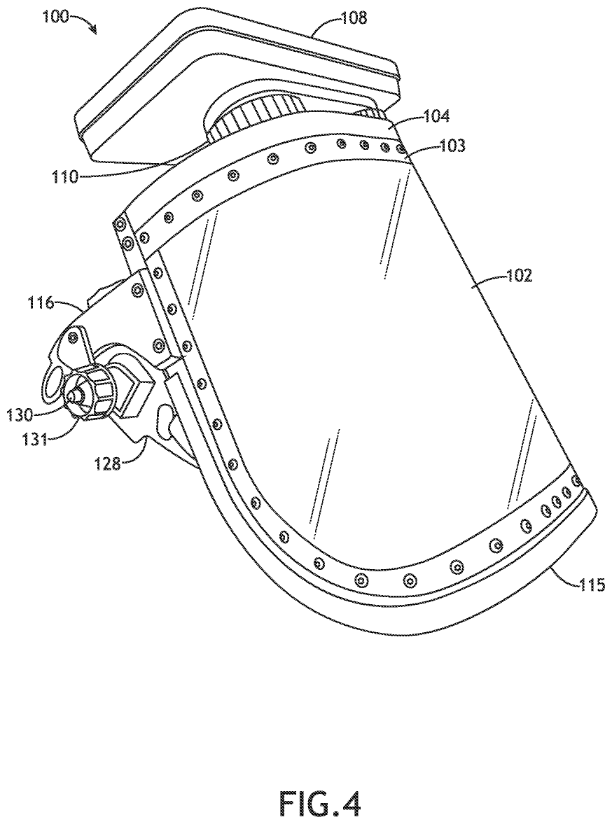

The face shield frame 103 may be pivotally coupled to the headgear 128 so that it can conveniently be switched from the raised inactive configuration to the lowered active configuration, and vice versa. For example, the PAPF 100 may include one or more side members 116 extending from the face shield frame 103, and the headgear 128 may include one or more pivots 130 that connect the side members 116 to the headgear 128. Preferably, as shown in FIGS. 3 and 4, the PAPF 100 includes at least one side member 116 on each side of the face shield frame 103, and the side members 116 are connected to respective pivots 130 on each side of the headgear 128. The headgear 128 may be removably coupled to the face shield frame 103 by the pivots 130 so that the components can be disassembled for sanitization and reassembled thereafter.

There are several methods for securing the face shield frame 103 in the raised or lowered configuration. For example, in the embodiments shown in FIGS. 3 and 4, the pivots 130 include threaded knobs 131 (or screws) that can be tightened by turning the knobs 131 (or screws) clockwise to secure the face shield frame 103 in the raised/lowered configuration or loosened by turning the knobs 131 (or screws) counterclockwise to allow movement of the face shield frame 103 about the pivots 130. In other embodiments, the tightening and loosening directions may be altered (e.g., clockwise to loosen and counterclockwise to tighten). Other methods of securing the face shield frame 103 in the raised/lowered configuration may include, but are not limited to, the use of pins, latches, magnetic couplings, and/or ratcheting pivots.

As shown in FIG. 3, the PAPF 100 may further include a battery holster 118 mounted to a side member 116 of the face shield frame 103, wherein the battery holster 118 is configured to hold an interchangeable battery pack 120 for the battery powered fan assembly 104. Alternatively, the battery holster 118/battery pack 120 can be mounted to a portion of the headgear 128 (e.g., like the system shown in FIGS. 8 through 10). In other embodiments, a rechargeable/replaceable battery can be integrated within the battery powered fan assembly 104. However, the use of an externally located interchangeable battery (e.g., battery pack 120) provides certain advantages as it allows for quick battery replacement on the go so that the user experiences little to no downtime. In some embodiments, to balance weight across the user's head, the PAPF 100 may include a rear-mounted battery pack 120 on the headgear 128 or two battery packs 120, one on each side member 116 or on each side of the headgear 128. The battery pack 120 may be electrically coupled to the battery powered fan assembly 104 by one or more connectors (e.g., wires/cables 124, M-M, F-F, M-F, and/or F-M couplers 126, and so forth). The one or more connectors may be configured to interface with a power input port 122 (e.g., USB-A port, micro-USB port, USB-C port, coaxial port, or the like) of the battery powered fan assembly 104.

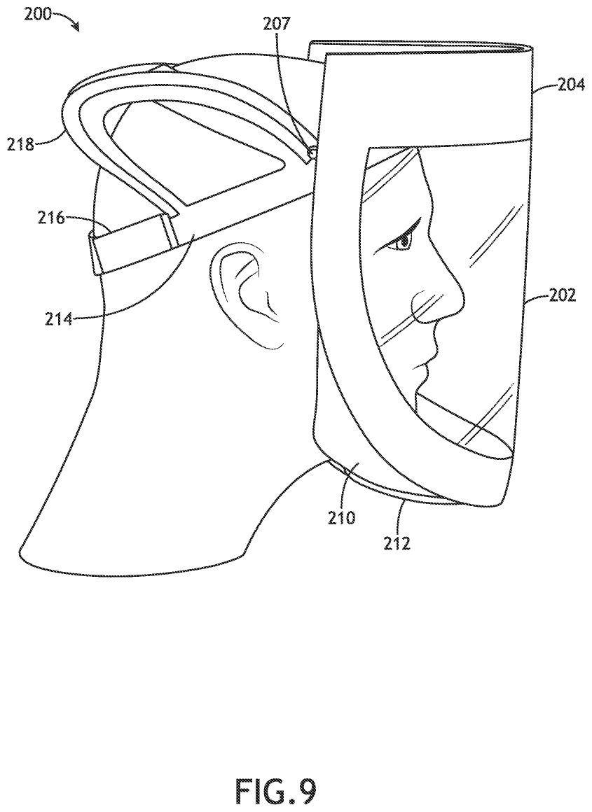

FIGS. 8 through 10 illustrate a PAPF 200 in accordance with another embodiment of this disclosure. As shown in FIGS. 8 and 9, the PAPF 200 includes a face shield 202 that is mounted to a face shield frame 204. For example, the face shield 202 may be held between inner and outer frame members of the face shield frame 204, or the face shield 202 may be secured to an inner or outer surface of the face shield frame 204 by a plurality of fasteners, adhesive (e.g., glue), or any other type of fastening mechanism.

In embodiments, the face shield 202 is designed to be larger than a typical user's face profile. For example, the face shield 202 may have a width greater than the user's ear-to-ear distance and a length greater than the user's forehead-to-chin distance. To achieve proper dimensions the face shield can be designed using (National Institute for Occupational Safety and Health) NIOSH Anthropometric Data and ISO Digital Headforms, published by The National Personal Protective Technology Laboratory (NPPTL), updated Apr. 24, 2020.

The PAPF 200 further includes a neck shroud 210 that is coupled to the face shield frame 204. In embodiments, the neck shroud 210 extends from a lower edge of the face shield frame 204 to seal gaps between a user's head and the face shield frame 204. The neck shroud 210 may include one or more filtered exhaust ports 212 that are configured to expel air from a region behind the face shield 202. In some embodiments, each filtered exhaust port 212 comprises an opening (or transmissive fabric) that includes or is overlaid by a filter for source control. The exhaust port filter material may include, but is not limited to, surgical mask material or any other ASTM level 3 filter material.

The neck shroud 210 may be removably coupled to the face shield frame 204 so that the neck shroud 210 and face shield frame 204 can be disassembled for sanitization and reassembled thereafter. In some embodiments, the neck shroud 210 is removably coupled to the face shield frame 204 by an edge guard that extends along the lower edge of the face shield frame 204. The edge guard may be configured to grasp an upper edge of the neck shroud 210 so that it is held tightly between the edge guard and the lower edge of the face shield frame 204.

In some embodiments, the neck shroud 210 is configured to be coupled to or interfaced with a full body PPE suit. For example, the neck shroud 210 may include one or more fasteners (e.g., buttons, buttonholes, snap fasteners, clasps, zipper elements, VELCRO elements, etc.) that are configured to secure a lower edge of the neck shroud 210 to a collar of a full body PPE suit. Alternatively, the neck shroud 210 may be coupled to the full body PPE suit by simply nesting the lower edge of the neck shroud within the collar of the full body PPE suit.

The PAPF 200 further includes a battery powered fan assembly 206 mounted above the face shield frame 204. The battery powered fan assembly 206 may include any type of electric fan or blower. For example, the battery powered fan assembly 206 may include at least one electric motor that drives a respective fan propeller. In some embodiments, the battery powered fan assembly 206 includes a VR headset fan or similar.

The battery powered fan assembly 206 may be mounted within a compartment 205 that is defined by or coupled to an upper edge of the face shield frame 204. In some embodiments, the battery powered fan assembly 206 is in its own compartment that is removably coupled to the upper edge of the face shield frame 204 by one or more fasteners (e.g., screws, bolts, latches, friction-fit couplings, etc.) so that the components can be disassembled for sanitization and then reassembled thereafter.

The battery powered fan assembly 206 is configured to direct ambient air through an air filter 208 before the air is blown into a region behind the face shield 202. In this manner, filtered air is constantly provided to the user. The air flows through the region behind the face shield 202 is eventually released through the one or more exhaust ports 212. The air filter 208 may be located at or adjacent to an air intake vent at the top of the PAPF 200. The battery powered fan assembly 206 may be located between the air filter 208 and the region behind the face shield 202 so that only filtered air enters the battery powered fan assembly 206. This configuration prevents the battery powered fan assembly 206 from being contaminated during use.

In embodiments, the battery powered fan assembly 206 is configured to suction air through the air filter 208 and then blow the air into the region behind the face shield 202 through one or more vents. In some embodiments, the vents have a protective covering with small enough openings to prevent the user's hair from being caught in the battery powered fan assembly 206.

The air filter 208 is preferably an N95 level or other HEPA filter with splash protection. In some embodiments, the air filter 208 is pleated to improve airflow through the system. The battery powered fan assembly 206 and the air filter 208 may be configured to provide airflow of at least 6 cfm; although less airflow may be appropriate in certain embodiments.

The air filter 208 may be located directly above the battery powered fan assembly 206. For example, the air filter 208 may be mounted within the same compartment 205 as the battery powered fan assembly 206. Alternatively, the air filter 208 may be mounted within a separate compartment that is coupled to the compartment 205 of the battery powered fan assembly 206 by one or more air conduits. The battery powered fan assembly 206 may be configured to generate an operational sound output below 50 dB; although higher noise output may be appropriate in certain embodiments.

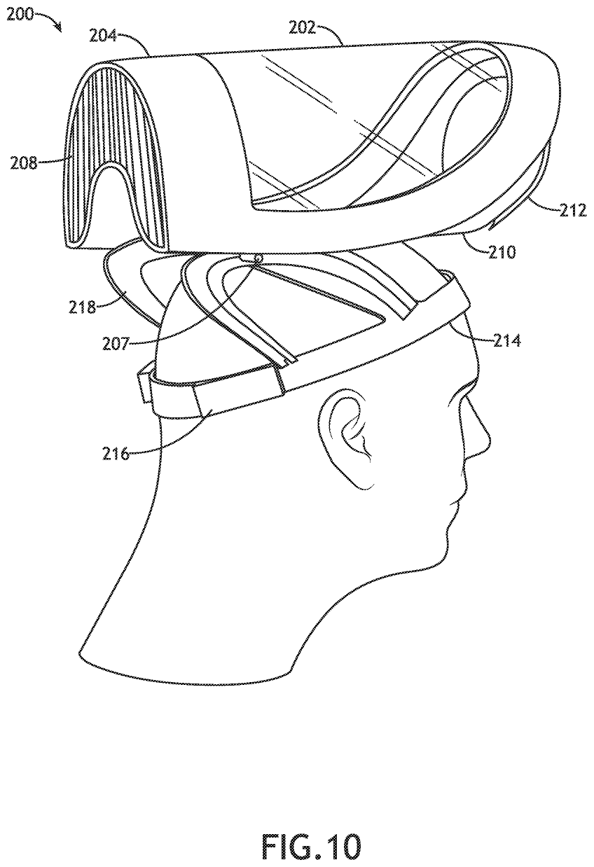

The PAPF 200 further includes a headgear 214 configured to support the PAPF components on the user's head. In embodiments, the headgear 214 includes a head strap configured to wrap around the back of the user's head. The headgear 214 preferably wraps entirely around the user's head so that it can hold the face shield frame 204 in a lowered configuration for active use (e.g., as shown in FIG. 9) or in a raised configuration when the PAPF 200 is inactive (e.g., as shown in FIG. 10). The headgear 214 may further include an additional strap configured to extend across a top of the user's head for added support. The user can easily don the PAPF 200 by securing the headgear 214 to the user's head. The headgear 214 may include elastic and/or adjustable straps (e.g., adjustable belts) to accommodate different head sizes.

The PAPF 200 is also made from lightweight materials to make it easy to wear for extended periods of time. In preferred embodiments, the PAPF 200 weighs less than 1.5 pounds; although heavier weight materials, hence a heavier PAPF 200, may be appropriate for certain embodiments (e.g., for use in very harsh environments).

The face shield frame 204 may be pivotally coupled to the headgear 214 so that it can conveniently be switched from the raised inactive configuration to the lowered active configuration, and vice versa. For example, the PAPF 200 may include one or more side bearings 207 (e.g., pins, rollers, dowels, or the like) extending from the face shield frame 204, and the headgear 214 may include one or more curved rails 218 that connect the one or more side bearings 207 to the headgear 214. Preferably, the PAPF 200 includes at least one side bearing 207 on each side of the face shield frame 204, and the side bearings 207 are connected to respective rails 218 on each side of the headgear 214. The side bearings 207 are used to actuate (e.g., slide) the face shield frame 204 along the curved rails 218 in order to pivot the face shield frame 204 from a raised inactive configuration to a lowered active configuration, and vice versa. The headgear 214 may be removably coupled to the face shield frame 204 by the curved rails 218 and bearings 207 so that the components can be disassembled for sanitization and reassembled thereafter.

The PAPF 200 may further include one or more interchangeable battery packs 216 for the battery powered fan assembly 206. The battery packs 216 can be mounted to a portion of the headgear 214, or alternatively to a portion of the face shield frame 204. In other embodiments, a rechargeable/replaceable battery can be integrated within the battery powered fan assembly 206. However, the use of externally located interchangeable battery (e.g., battery pack 216) provides certain advantages as it allows for quick battery replacement on the go so that the user experiences little to no downtime. In some embodiments, to balance weight across the user's head, the PAPF 200 may include a rear-mounted battery pack 216 on the headgear 214 or two battery packs 216, one on each side of the face shield frame 204 or on each side of the headgear 214. The battery packs may be electrically coupled to the battery powered fan assembly 206 by one or more connectors (e.g., wires/cables, M-M, F-F, M-F, and/or F-M couplers, and so forth). The one or more connectors may be configured to interface with a power input port (e.g., USB-A port, micro-USB port, USB-C port, coaxial port, or the like) of the battery powered fan assembly 206.

Specific embodiments of PAPF 100 and PAPF 200 have been described with reference to FIGS. 1 through 7 and FIGS. 8 through 10, respectively. Nevertheless, as previously noted herein, two or more embodiments (or portions thereof) can be combined to achieve an additional embodiment of the PAPF 100/200 that is not necessarily shown or described with reference to one drawing. As such, the drawings should not be construed as restrictive of any particular embodiment and are intended instead as visual aids to help describe configurations of certain components that may apply to multiple embodiments of the PAPF 100/200. For example, PAPF 100 may be modified to include the rails and bearings of PAPF 200 in place of its securable pivots, or PAPF 200 may be modified to include the separate compartments of PAPF 100 for its battery powered fan assembly and air filter. Similarly, any other components/configurations from PAPF 100 may be exchanged for components/configurations from PAPF 200, and vice versa, to the extent that such change does not affect the operability of the PAPF 100/200.

Although the technology has been described with reference to the embodiments illustrated in the attached drawing figures, equivalents may be employed, and substitutions may be made herein without departing from the scope of the technology as recited in the claims. Components illustrated and described herein are examples of devices and components that may be used to implement the embodiments of the present invention and may be replaced with other devices and components without departing from the scope of the invention. Furthermore, any dimensions, degrees, and/or numerical ranges provided herein are to be understood as non-limiting examples unless otherwise specified in the claims.

1: A powered air purifying faceshield (PAPF), comprising:
a headgear;
a face shield frame pivotally coupled to the headgear;
a face shield mounted to the face shield frame;
a battery powered fan assembly mounted above the face shield frame, the battery powered fan assembly being configured to direct ambient air through an air filter before the air is blown into a region behind the face shield; and
a neck shroud coupled to the face shield frame, the neck shroud including one or more filtered exhaust ports.

2: The PAPF of claim 1, wherein the headgear comprises a head strap configured to wrap around a back of a user's head.

3: The PAPF of claim 2, wherein the headgear further comprises an additional strap configured to extend across a top of the user's head for added support.

4: The PAPF of claim 1, further comprising one or more side members extending from the face shield frame, wherein the face shield frame is pivotally coupled to the headgear by one or more securable pivots that connect the one or more side members to the headgear.

5: The PAPF of claim 4, wherein the one or more securable pivots are configured to hold the face shield frame in a raised inactive configuration or in a lowered active configuration.

6: The PAPF of claim 1, further comprising one or more side bearings extending from the face shield frame, wherein the face shield frame is pivotally coupled to the headgear by one or more curved rails that connect the one or more side bearings to the headgear.

7: The PAPF of claim 6, wherein the one or more side bearings are used to actuate the face shield frame along the one or more curved rails in order to pivot the face shield frame from a raised inactive configuration to a lowered active configuration, and vice versa.

8: The PAPF of claim 1, wherein the neck shroud is configured to be coupled or interfaced to a full body personal protective equipment (PPE) suit.

9: The PAPF of claim 1, further comprising a battery holster mounted to a side member of the face shield frame or to a portion of the headgear, wherein the battery holster is configured to hold an interchangeable battery pack for the battery powered fan assembly.

10: The PAPF of claim 9, further comprising one or more connectors configured to electrically couple the interchangeable battery pack to the battery powered fan assembly.

11: The PAPF of claim 1, wherein the battery powered fan assembly is mounted within a compartment that is defined by or coupled to an upper edge of the face shield frame.

12: The PAPF of claim 11, wherein the air filter is mounted within a separate compartment that is coupled to the compartment of the battery powered fan assembly by one or more air conduits.

13: The PAPF of claim 12, wherein the one or more air conduits comprise threaded couplings for securing the separate compartment of the air filter to the compartment of the battery powered fan assembly.

14: The PAPF of claim 11, wherein the air filter is mounted within the same compartment as the battery powered fan assembly.

15: The PAPF of claim 1, wherein the headgear and the neck shroud are removably coupled to the face shield frame such that they can be disassembled for sanitization and reassembled thereafter.

16: The PAPF of claim 1, wherein the air filter is a high efficiency particulate air (HEPA) filter with splash protection.

17: The PAPF of claim 1, wherein the one or more filtered exhaust ports include American Society for Testing Materials (ASTM) level 3 filter material.

18: The PAPF of claim 1, wherein the battery powered fan assembly and the air filter are configured to provide airflow of at least 6 cubic feet per minute (cfm).

19: The PAPF of claim 1, wherein the battery powered fan assembly has an operational sound output below 50 decibels (dB).

20: The PAPF of claim 1, wherein the PAPF weighs less than 1.5 pounds.
